(19) United States
(12) Patent Application Publication
(10) Pub. No.: US 2026/0310850 A1
(43) Pub. Date: Oct. 8, 2026

(54) METHOD AND SYSTEM FOR PREDICTING FIXATION PERFORMANCES OF A SCREW INSERTED INTO A MATERIAL BLOCK

(71) Applicant: Spinologics Inc., Montreal (CA)

(72) Inventors: David BENOIT, Montreal (CA); Franck LE NAVÉAUX, Montreal (CA); Julien CLIN, Montreal (CA); Loïc DEGUELDRE, Montreal (CA); Bahe HACHEM, Montreal (CA)

(73) Assignee: Spinologics Inc., Montreal, QC (CA)

(21) Appl. No.: 19/483,458

(22) PCT Filed: May 10, 2024
(86) PCT No.: PCT/CA2024/050631

§ 371 Date: Nov. 11, 2025

Related U.S. Application Data
(60) Provisional application No. 63/466,058, filed on May 12, 2023.

(51) Int. Cl. G06F 30/20; G06F 30/12; G06F 119/14

(52) U.S. Cl. CPC G06F 30/20; G06F 30/12; G06F 2119/14

(57) ABSTRACT

Methods, systems, and techniques for predicting fixation performances of a screw inserted into a material block. The method comprising: receiving a three-dimensional geometry of the screw; creating a computer model of the screw and the material block taking into account the three-dimensional geometry, wherein the screw is inserted into the material block in said computer model; simulating a load being applied on the screw using the computer model; and computing fixation performances using the simulation.

### CROSS-REFERENCE TO RELATED APPLICATIONS

This application claims priority from U.S. provisional patent application No. 63/466,058 filed on May 12, 2023, which is incorporated herein by reference in its entirety.

### FIELD

The present disclosure is directed at methods, systems, and techniques for predicting fixation performances of a screw inserted into a material block.

### BACKGROUND

Accurately predicting fixation performances of a screw inserted into a material block is a problem faced by many industries as small changes in the design of the screw or in material properties of the block material may lead to big variations in performances of the screw-material block construct.

Achieving optimal screw fixation performance is crucial in many fields of application (e.g., construction), and notably in orthopedics as it directly impacts the stability and strength of bone fixation, whether used alone or in combination with plate and rod systems. Screw loss of fixation, known as screw loosening or pullout, is a common complication that can arise during or following surgery. In spinal surgery for instance, this complication occurs in about 10% of patients (Bredow et al., 2016; Soroceanu et al., 2015), with a higher incidence in patients with osteoporosis (Galbusera et al., 2015). The consequences of this complication can include significant morbidity and decreased patient quality of life, as well as the need for revision surgeries.

An approach to evaluate the mechanical fixation strength of orthopedic screws is to conduct screw pullout tests in cadaveric bone. However, the mechanical properties of bone can vary significantly based on factors such as age, gender, and health, which can make it difficult to generalize findings and compare results across different studies. Synthetic bones (Sawbones™, Pacific Research Laboratories, WA, USA), are commonly used to evaluate the fixation strength of orthopedic screws as they allow for standardized testing conditions and consistent mechanical properties. The polyurethane foam of the synthetic bone is designed to mimic the mechanical properties of trabecular bone (Calvert, Trumble, Webster, & Kirkpatrick, 2010) and has been shown to be a reliable and accurate predictor of screw pullout force in bone (Nagaraja & Palepu, 2016). Furthermore, the polyurethane foam is considered a standard material for mechanical tests involving orthopedic devices, according to the ASTM F1839 protocol.

Considerable efforts are being made towards improving the mechanical performance of orthopedic screws, but the iterative process of designing, prototyping, testing and refining screw designs is costly and time-consuming which may limit engineers to reach an optimal design. Furthermore, the evaluation of the effect of screw design features on performance using experimental testing is limited by the inherent variability associated with conducting multiple rounds of tests at different time points, which can lead to complications in the analysis of results.

The Chapman's analytical formula (Chapman et al., 1996) has been proposed as an alternative approach to estimate screw pullout forces by considering screws' main geometric features such as major diameter, thread depth and thread pitch. This Formula is utilized in the FDA guidance: “Orthopedic Non-Spinal Metallic Bone Screws and Washers—Performance Criteria for Safety and Performance Based Pathway” as an analytical way to estimate and qualify screw design. However, this formula has multiple limitations, as it only considers a limited set of screw geometrical features which can be subject to varying interpretations by different users.

Therefore, there is a need to develop new methods of predicting fixation performances of a screw inserted into a material block.

### SUMMARY

According to a first aspect, a method is provided for predicting fixation performances of a screw inserted into a material block, the method being implemented on an electronic device with one or more processors and a memory. The method comprises receiving a three-dimensional geometry of the screw; creating a computer model of the screw and the material block taking into account the three-dimensional geometry, wherein the screw is inserted into the material block in said computer model. The method also comprises simulating a load being applied on the screw using the computer model; and computing fixation performances using the simulation.

In one embodiment, the method further comprises outputting the computed fixation performances.

In an example embodiment, the computed fixation performances comprise one or more of:
a load at failure;
a load-displacement curve representing a displacement of the screw relative to the material block when the load is applied on the screw;
the displacement of the screw at failure;
stiffness of the screw-block material construct;
a screw-block material construct failure mode;
a strain distribution plot;
a deflection distribution plot;
a cross sectional visualization of screw-block material interface; or
a stress distribution plot.

In an example embodiment, the method further comprises, prior to creating the computer model, receiving information on the material block comprising one or more of: a position and orientation where the screw is to be inserted, a depth at which the screw is to be inserted, dimensions of the material block, a density of the material block, and homogeneity of the material block.

In an example embodiment, when the material block is heterogeneous, the method further comprises receiving one or more of a thickness and density of layers of the material block.

In an example embodiment, creating the computer model of the screw and the material block comprises: updating a model of the material block taking into account the material block information; generating the block material using the model of the material block and the three-dimensional geometry, position and orientation of the screw; and generating the computer model of the screw and the material block.

In an example embodiment, the material block model is calibrated to mimic the material block response to loading modes comprising one or more of: torsion, shear, tension, indentation, compression, or any combination thereof.

In an example embodiment, the material block comprises a synthetic bone.

In an example embodiment, the computer model comprises boundary conditions defined to simulate the load as required by an ASTM F543 protocol.

According to a second aspect, a non-transitory medium is provided for storing computer-readable instructions which, when read and executed by at least one processor of a computing device, implement: a Graphical User Interface (GUI) functional block configured for implementing a computerized GUI that allows a user of the computing device to upload a three-dimensional screw geometry and displays a depiction of the screw; a processing functional block and a data file generator. The processing functional block is configured for sending a signal to query a processor to: create a computer model of the screw and the material block taking into account the three-dimensional screw geometry, wherein the screw is inserted into the material block in said computer model; simulate a load being applied on the screw using the computer model; and compute fixation performances using the simulation. The data file generator is configured for generating a data file comprising information obtained from the simulation, said information comprising the computed fixation performances.

In an example embodiment, the computed fixation performances comprise one or more of:
a load at failure;
a load-displacement curve representing a displacement of the screw relative to the material block when the load is applied on the screw;
the displacement of the screw at failure;
stiffness of the screw-block material construct;
a screw-block material construct failure mode;
a strain distribution plot;
a deflection distribution plot;
a cross sectional visualization of screw-block material interface; or
a stress distribution plot.

In an example embodiment, the computerized GUI allows the user to fill-in material block information comprising one or more of: a position and orientation where the screw is to be inserted, a depth at which the screw is to be inserted, dimensions of the material block, a density of the material block, and homogeneity of the material block.

In an example embodiment, when information on homogeneity of the material block indicates a heterogeneous material, the computerized GUI allows the user to fill-in one or more of a thickness and density of layers of the material block.

In an example embodiment, the user is allowed to download the data file, wherein the data file comprises one or more of the three-dimensional screw geometry, the computed fixation performances, the position and orientation where the screw is inserted, the depth at which the screw is inserted, dimensions of the material block, the density of the material block, the homogeneity of the material block, and the thickness and density of layers of the material block.

According to another aspect, there is provided a non-transitory computer readable medium having stored thereon computer program code that is executable by a processor and that, when executed by the processor, causes the processor to perform the foregoing method.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

In the accompanying drawings, which illustrate one or more example embodiments:

FIGS. 1 & 2 depict a method for predicting fixation performances of a screw inserted into a material block, according to an example embodiment.

FIG. 3 depicts example fixation performances computed in accordance with the method of FIGS. 1 & 2.

FIG. 4 depicts key steps of the method of FIGS. 1 & 2 being applied on an example screw geometry, according to an example embodiment.

FIG. 5 depicts an example computer system that may be used to predict fixation performances of a screw inserted into a material block, according to an example embodiment.

FIG. 6 depicts a sample of orthopedic screws included in a validation study performed according to an example embodiment.

FIG. 7 depicts a value of a plurality of screw geometric features and a profile view of a plurality of screw threads used in the validation study performed according to an example embodiment.

FIG. 8 shows a picture of the experimental setup for screw insertion (left) and screw pullout (right) used in the validation study performed according to an example embodiment.

FIG. 9 depicts a graph of grade 20 PCF stress strain relations.

FIG. 10 depicts a plurality of experimental (dark) and simulated (light) screw pullout force-displacement curves computed according to an example embodiment.

FIG. 11 depicts two simulated and the corresponding two experimental failure modes of the synthetic bone computed according to an example embodiment.

FIG. 12 depicts a comparison between measured and predicted screw pullout forces computed according to an example embodiment.

FIG. 13 depicts the mean error prediction and 95% confidence intervals computed according to an example embodiment.

FIG. 14 depicts the correlation between experimental and predicted pullout forces computed according to an example embodiment.

### DETAILED DESCRIPTION

The terminology used herein is in accordance with definitions set out below.

By “about”, it is meant that a value or data disclosed herein can vary within a certain range depending on the margin of error of the method or device used to obtain or evaluate such value or data. A margin of error of 10% of such value or data is generally accepted. It should also be noted that terms of degree such as “substantially” and “approximately” as used herein mean a reasonable amount of deviation of the modified term such that the end result is not significantly changed. These terms of degree may also be construed as including a deviation of the modified term if this deviation would not negate the meaning of the term it modifies.

The description which follows, and the embodiments described therein are provided by way of illustration of an example of particular embodiments of principles and aspects of the present invention. These examples are provided for the purposes of explanation and not of limitation, of those principles of the invention. In the description that follows, like parts and/or steps are marked throughout the specification and the drawing with the same respective reference numerals or signs.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g., a pushbutton keyboard, mouse, a touchscreen, and the like), and at least one output device (e.g., a display screen, a printer, a wireless radio, and the like) depending on the nature of the device.

It should also be noted that there may be some elements that are used to implement at least part of one of the embodiments described herein that may be implemented via software that is written in a high-level computer programming language such as object-oriented programming or script-based programming. Accordingly, the program code may be written in Java, Swift/Objective-C, C, C++, Javascript, Python, SQL or any other suitable programming language and may comprise modules or classes, as is known to those skilled in object-oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage. The computer program product may also be distributed in an over-the-air or wireless manner, using a wireless data connection.

The term “software application” or “application” refers to computer-executable instructions, particularly computer-executable instructions stored in a non-transitory medium, such as a non-volatile memory, and executed by a computer processor. The computer processor, when executing the instructions, may receive inputs and transmit outputs to any of a variety of input or output devices to which it is coupled. Software applications may include mobile applications or “apps” for use on mobile devices such as smartphones and tablets or other “smart” devices.

A software application can be, for example, a monolithic software application, built in-house by the organization and possibly running on custom hardware; a set of interconnected modular subsystems running on similar or diverse hardware; a software-as-a-service application operated remotely by a third party; third party software running on outsourced infrastructure, etc. In some cases, a software application also may be less formal, or constructed in ad hoc fashion, such as a programmable spreadsheet document that has been modified to perform computations for the organization's needs.

Software applications may be deployed to and installed on a computing device on which it is to operate. Depending on the nature of the operating system and/or platform of the computing device, an application may be deployed directly to the computing device, and/or the application may be downloaded from an application marketplace.

As aforesaid, according to a first aspect, a method is provided for predicting fixation performances of a screw inserted into a material block, the method being implemented on an electronic device with one or more processors and a memory. The method comprises receiving a three-dimensional geometry of the screw; creating a computer model of the screw and the material block taking into account the three-dimensional geometry, wherein the screw is inserted into the material block in said computer model. The method also comprises simulating a load being applied on the screw using the computer model; and computing fixation performances using the simulation.

Computational modeling and simulation (CM&S) can effectively address the issues raised above and can offer an alternative method to predict the fixation strength, and performances in general, of screws.

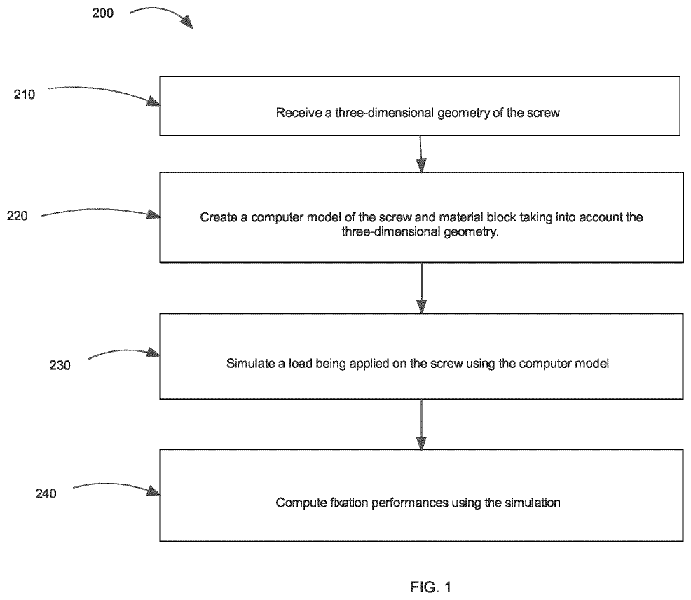

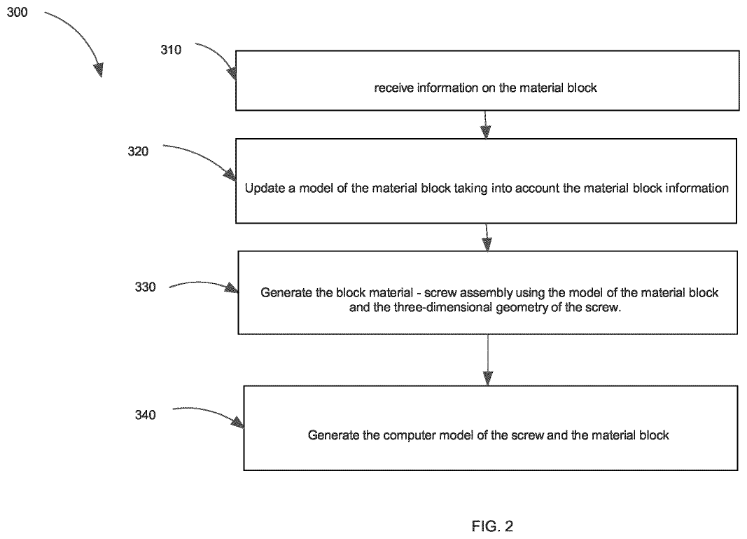

In accordance with the present disclosure, systems and methods are disclosed for predicting fixation performances of a screw inserted into a material block. FIG. 1 shows a method 200 for predicting fixation performances of a screw inserted into a material block wherein method 200 is implemented on an electronic device with one or more processors and a memory.

Method 200 comprises receiving 210 a three-dimensional geometry of the screw. In some embodiments, the three-dimensional geometry of the screw may be received as CAD file.

Then, a computer model of the screw and the material block that takes into account the three-dimensional geometry of the screw is created 220. The screw is inserted into the material block in said computer model (i.e., the material block surrounds at least a portion of the screw). Creation of the computer model of the screw and the material block is further described below in accordance with FIG. 2.

Upon creation 220 of the computer model, method 200 further goes to simulating 230 a load being applied on the screw using the computer model.

The load may be applied on the screw as a force and/or as a moment. As will be appreciated by the skilled reader, the force, or combinations thereof, may be applied to various parts of the screw and in any direction in three-dimensional space. Moreover, the moment may represent the measure of any force, or combinations thereof, to cause the screw to rotate about a specific point or axis. In one embodiment, the load may be applied as pullout force to extract the screw from the block material.

Further, method 200 goes to computing 240 fixation performances using the simulation.

In some instances, method 200 may further comprise outputting 250 the computed fixation performances.

Depending on the application, the computed fixation performances may comprise a load at failure. Failure occurs when there is a rupture of the block material around the screw. In some embodiments, the rupture needs to be complete to be considered as failure. In other embodiments, a threshold percentage of the material block surrounding the screw needs to be ruptured to reach failure. In some instances, a rapid decline in the force-displacement curve response is indicative of failure. A person skilled in the art would already acknowledge that depending on sensitivity of the end application, failure may be defined a plurality of ways. Anyhow, the load at failure is the load applied on the screw when rupture of the block material around the screw occurs.

In some embodiments, the computed fixation performances may comprise one or more of:
a load-displacement curve representing a displacement of the screw relative to the material block when the load is applied on the screw;
the displacement of the screw at failure;
stiffness of the screw-block material construct;
a screw-block material construct failure mode;
a strain distribution plot;
a deflection distribution plot;
a cross sectional visualization of screw-block material interface; and
a stress distribution plot.

The screw-block material construct failure mode refers to the specific way in which the material block breaks or fractures under the load applied to the screw. The mode of failure may help to understand the screw-block material behavior. In the example where the load is a pullout force applied on the screw, the mode of failure of the material block when the screw is pulled out refers to the specific way in which the material block breaks or fractures under the force of a screw being pulled out.

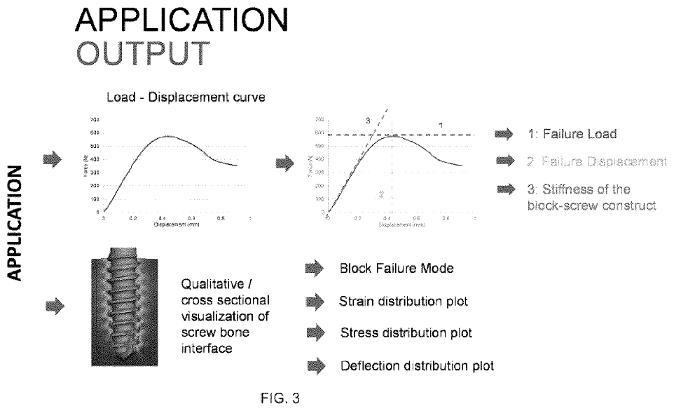

FIG. 3 shows a load-displacement curve, stiffness of the screw-block material construct, the failure load, the failure displacement, and a cross sectional visualization of screw bone interface computed for an example screw-block material construct in accordance with the method 200.

Back to FIG. 2, wherein a method 300 for creating the computer model of the screw and the material block is shown. Method 300 may start with receiving 310 information on the material block. The information on the material block may comprise one or more of a position and orientation where the screw is to be inserted, a depth at which the screw is to be inserted, dimensions of the material block, a density of the material block, and homogeneity of the material block.

When the material block is heterogeneous, the information on the material block may further comprise one or more of a thickness and density of layers of the material block.

Once the information on the material block is received 310, method 300 may go to updating 320 a model of the material block taking into account the material block information.

In embodiments where the information on the material block does not change, steps 310 and 320 may not be performed.

In one embodiment (not shown), geometrical features of the screw comprising one or more of a screw length, a thread profile comprising a thread length and/or a thread diameter, a pitch, and a core diameter are received.

Next, method 300 goes to generate 330 the block material around the screw using the model of the material block, the geometrical feature of the screw and the three-dimensional geometry of the screw.

The computer model of the screw and the material block is generated 340 using the generated block material around the screw 330. Generating 340 the computer model of the screw and the material block 340 is known in the art and may comprise generation of mesh representation of the screw-material block construct, applying boundary conditions to the screw-material block construct, etc.

With respect to the material block model, the material block model is calibrated to mimic the material block response to loading modes comprising one or more of torsion, shear, tension, indentation, compression, or any combination thereof. In this way, the response of the material block to mechanical loads is already known.

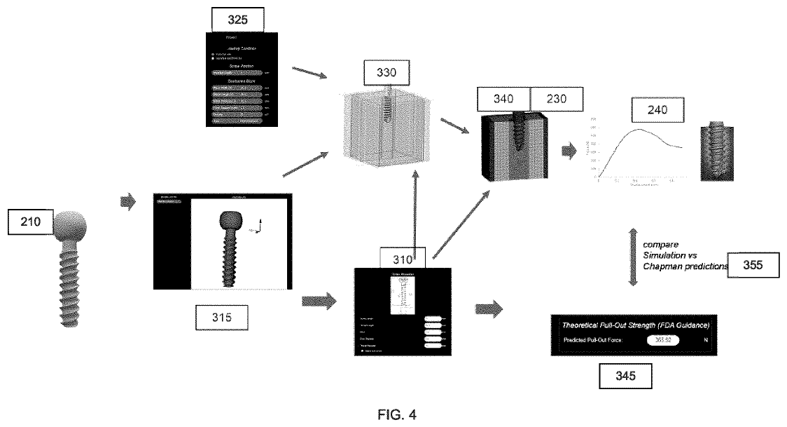

FIG. 4 depicts key steps of method 200 and method 300 being applied on an example screw geometry, according to an example embodiment. For instance, FIG. 4 shows a three-dimensional geometry of the screw that is received 210. The three-dimensional geometry of the screw may be obtained as a computer assisted design (CAD). The three-dimensional geometry of the screw is uploaded 315 into a graphical user interface. Following, geometrical features of the screw comprising one or more of a screw length, a thread profile comprising a thread length and/or a thread diameter, a pitch, and a core diameter are received 310.

In one embodiment, the graphical user interface queries the user to fill-in the geometrical features.

In another embodiment, the geometrical features are automatically computed using the three-dimensional screw geometry.

In yet another embodiment, the geometrical features are automatically computed using the three-dimensional screw geometry and the user is further allowed to confirm and/or change the computed geometrical features.

Next, the graphical user interface queries the user to fill-in 325 information on the material block. Said information on material block may comprise one or more of a position and orientation where the screw is to be inserted, a depth at which the screw is to be inserted, dimensions of the material block, a density of the material block, and homogeneity of the material block. When the material block is heterogeneous, the information on the material block may further comprise one or more of a thickness and density of layers of the material block. This material block information allows updating 320 a previously trained model of the material block.

Provided the updated model of the material block, the three-dimensional geometry of the screw, and the geometrical features of the screw, the screw-material block construct is generated. In some embodiments, the block material is generated 330 around the screw to form the screw-material block construct.

In an example embodiment, a construct portion surrounding an interface between the screw and the material block is defined in the screw-material block construct.

In some embodiments, the geometrical features of the screw may be used to fine tune the screw-material block construct. Fine tuning may for instance be used to define the geometrical features of the construct portion.

Next, the computer model of the screw and the material block is generated and boundary conditions are applied thereon. In some embodiments, the computer model is based on the finite element method. In some embodiments, the material block is generated 340 using a finite element method where a mesh is generated for the screw material block construct and where boundary conditions are applied thereon.

In some embodiments, the geometrical features of the screw may be used to fine tune the computer model of the screw and the material block.

Fine-tuning may for instance be used to define the size and geometry of the mesh of the construct portion, as a refined mesh at the construct portion allows accurately capturing the pullout force and the material block failure mode.

A simulation where a load is applied on the screw of the screw-material block construct is then run 230.

The geometrical features of the screw may alternatively or additionally be used to fine tune analysis parameters of the simulation.

Next, fixation performances are computed 240 using the simulation.

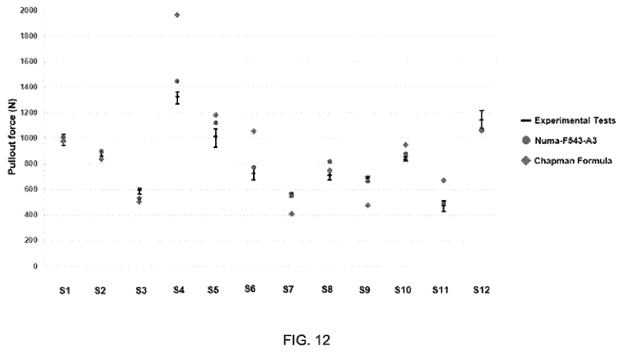

In one embodiment, the material block is a synthetic bone and theoretical pull-out strength of the Chapman formula are computed 345 based on the geometrical features of the screw. The Chapman formula is discussed in detail below with respect to FIGS. 12 to 14. In this embodiment, the graphical user interface may allow the user to compare 355 the theoretical pull-out strength to the computed fixation performances.

In a preferred embodiment, the material block is a synthetic bone and the material block model is a finite element model of said synthetic bone that allows accurately simulating the interaction between the screw and the synthetic bone material.

In this preferred embodiment, the computer model comprises boundary conditions defined to simulate the load as required by an ASTM F543 protocol.

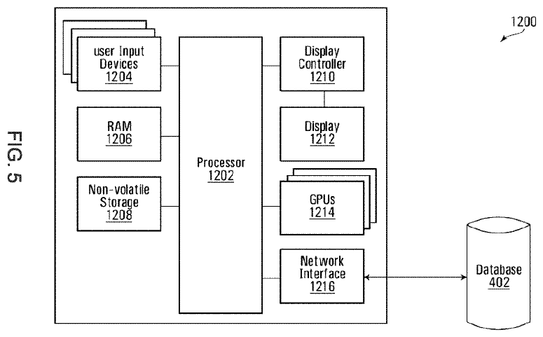

The methods described herein may be implemented at least in part by one or more computer systems, such as the computer system 1200 depicted in FIG. 5 which shows a block diagram of an example computer system 1200 comprising a processor 1202 that controls the system's 1200 overall operation. The processor 1202 is communicatively coupled to and controls subsystems comprising user input devices 1305, which may comprise any one or more user input devices such as a keyboard, mouse, touch screen, and Graphical User Interface (GUI); random access memory (“RAM”) 1206, which stores computer program code that is executed at runtime by the processor 1202; non-volatile storage 1208 (e.g., a solid state drive or magnetic spinning drive), which stores the computer program code loaded into the RAM 1305 for execution at runtime and other data; a display controller 1210, which may be communicatively coupled to and control a display 1212; graphical processing units (“GPUs”) 1214, used for parallelized processing and a network interface 1216, which facilitates network communications with a network and other devices that may be connected thereto.

The Graphical User Interface (GUI) functional block is configured for implementing a computerized GUI that allows a user of the computing device to upload a three-dimensional screw geometry and displays a depiction of the screw.

The computer system 1200 also comprises a database 402 that is communicatively coupled to the processor 1202 via the network interface 1216. The database 402 may be used to store multiple three-dimensional screw geometries, a model of the material block, and/or material block information.

The processing functional block 1202 may comprise any suitable processing unit such as a processor, microprocessor, artificial intelligence accelerator, or programmable logic controller, or a microcontroller (which comprises both a processing unit and a non-transitory computer readable medium), or system-on-a-chip (SoC). Examples of computer readable media that are non-transitory include disc-based media such as CD-ROMs and DVDs, magnetic media such as hard drives and other forms of magnetic disk storage, semiconductor based media such as flash media, random access memory (including DRAM and SRAM), and read only memory. As an alternative to an implementation that relies on processor-executed computer program code, a hardware-based implementation may be used. For example, an application-specific integrated circuit (ASIC), field programmable gate array (FPGA), or other suitable type of hardware implementation may be used as an alternative to or to supplement an implementation that relies primarily on a processor executing computer program code stored on a computer medium.

In accordance with FIG. 1 to FIG. 3, a non-transitory medium is provided for storing computer-readable instructions which, when read and executed by at least one processor of a computing device, implement a Graphical User Interface (GUI) functional block configured for implementing a computerized GUI that allows a user of the computing device to upload a three-dimensional screw geometry and displays a depiction of the screw. A processing functional block is also implemented and is configured for sending a signal to query a processor to: create a computer model of the screw and the material block taking into account the three-dimensional screw geometry, wherein the screw is inserted into the material block in said computer model; simulate a load being applied on the screw using the computer model; and compute fixation performances using the simulation as described with respect to methods 200 and 300. A data file generator is also implemented and is configured for generating a data file comprising information obtained from the simulation, said information comprising the computed fixation performances.

The computerized GUI may be configured to allow the user to fill-in material block information comprising one or more of a position and orientation where the screw is to be inserted, a depth at which the screw is to be inserted, dimensions of the material block, a density of the material block, a load direction on the screw and homogeneity of the material block.

The computerized GUI may be configured to allow the user to fill-in one or more of a thickness and density of layers of the material block for a heterogeneous material.

The computerized GUI may be further configured to allow the user to download the data file, wherein the data file comprises one or more of the three-dimensional screw geometry, the computed fixation performances, the position and orientation where the screw is inserted, the depth at which the screw is inserted, dimensions of the material block, the density of the material block, the homogeneity of the material block, and the thickness and density of layers of the material block.

In accordance with FIG. 1 to FIG. 3, there is provided a screw fixation performance prediction system, comprising: a processor; and a non-transitory computer-readable memory storing computer-executable instructions which, when executed by the processor, configure the system to perform the ongoing methods.

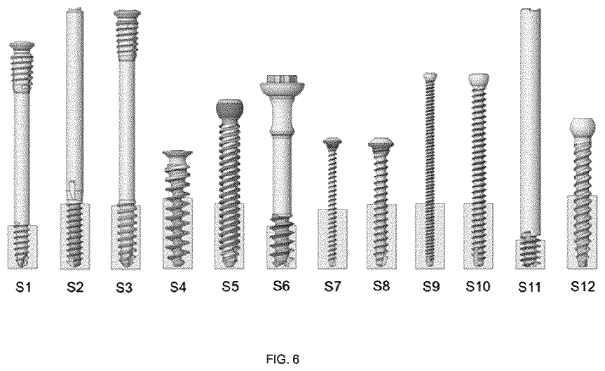

FIG. 6 shows a sample of screws included in a validation study. The validation study was conducted to compare the results of the ongoing methods with experimental results using Sawbones™ blocks. The validation study was in the orthopedic field. Hence, the tested screws belonged to the orthopedic field and the block material was a synthetic bone. The validation study also compared the results of the ongoing methods with the Chapman formula.

FIG. 6 shows twelve orthopedic screws (S1 to S12) selected to encompass a wide range of geometric features and designs among orthopedic screws, allowing an extensive assessment of the predictive capabilities of the method. As will be appreciated by the skilled reader, the present disclosure is not limited to the use of the twelve screws selected, nor is it limited to the use of twelve screws. The present disclosure may have been arrived at using other types of screws and other numbers of screws.

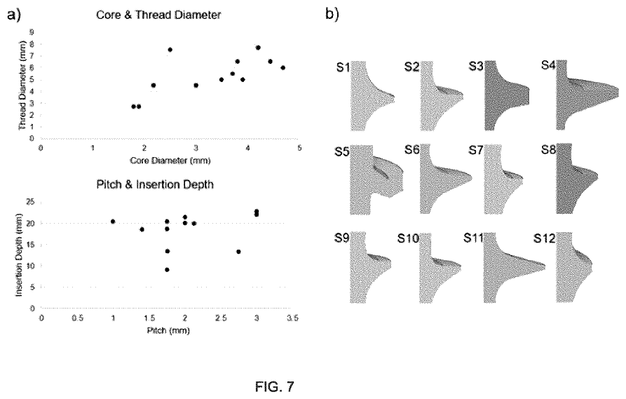

FIG. 7 shows that screws core and outer thread diameters varied from 1.8 to 4.69 mm, and 2.7 to 7.7 mm, respectively, while pitch varied from 1 to 3 mm. The thread profiles of the screws varied between standard buttress threads, variations of rounded V-thread, and fastener thread. S1, S2 and S12 screws had a tapered core diameter, while all other screws had a cylindrical core diameter. S4 screw required tapping a pilot hole before insertion, while all others were self-tapping. The gray boxes represent insertion depth of the screw in the material block.

### Experimental Testing

The protocol for evaluating the pullout strength of the screws was conducted in accordance with ASTM F543 guidelines. Solid material blocks of 20 PCF density were obtained from the manufacturer (Sawbones, Pacific Research Laboratories, WA, USA) and the real apparent density of the blocks was determined according to ASTM D1622 guidelines.

Pilot holes were drilled in the Sawbones blocks, with diameters slightly smaller than the core diameter of each respective screw. Screw insertion perpendicularly to the blocks was then carried out using a motorized torque test stand (Mark-10 TSTM-DC-1) at a rotation rate of 3 RPM (Error! Reference source not found.). A vertical force was initially applied on the screws to ensure it was properly engaged in the foam block and a 7 N axial load was maintained throughout the remainder of the insertion process. Insertion depth was measured after each insertion. To ensure the pullout results were not affected by potential temperature increase during screw insertion, awaiting period of 10 minutes was implemented before pullout (Fredericks & Nuckley, 2021).

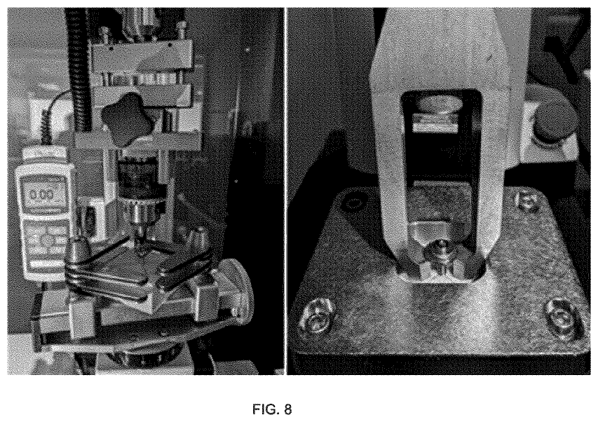

The screws were then axially pulled out of the sawbones block at a rate of 3 mm/min using a motorized tension test stand (Mark-10 ESM303) as the one shown in FIG. 8. Force-displacement curve was collected and the pullout strength of the screws was determined as the maximum force from the curves. Five pullout repetitions were conducted per screw to account for experimental variability, for a total of 60 pullout tests.

### Analytical Estimation Using the Chapman Formula

The Chapman formula was used to estimate the screw pull-out force in synthetic bone for each screw. To incorporate the screw thread shape, a thread shape factor (TSF) was defined:

$${F}_{pullout} = {S}_{shear} * A = {S}_{shear} * L * π * {D}_{major} * TSF$$

with,
Fₚᵤₗₗₒᵤₜ being the predicted pullout force (N);
Sₛₕₑₐᵣ being the material ultimate shear stress (MPa);
A being the thread shear area (mm²);
L being the axial thread length (mm) including only threads that have the nominal major diameter where complete purchase is expected (e.g., excluding the screw tip) of thread engagement in material;
Dₘₐⱼₒᵣ being the screw major diameter (mm); and
TSF being a Thread Shape Factor (dimensionless).

Wherein the Thread Shape Factor (TSF) was defined as:

$$TSF = ( \frac{1}{2} + \frac{1}{\sqrt{3}} * \frac{d}{p} )$$

with,
p being the thread pitch of the screw (mm); and
d being a thread depth of the screw (mm) defined as:

$$d = ( \frac{{D}_{major} - {D}_{minor}}{2} )$$

with, Dₘᵢₙₒᵣ being the minor (root) diameter (mm)

For each screw, the geometrical features considered in the equation were measured on the three dimensional screw geometry (e.g., the CAD model). The axial thread length was calculated by subtracting the screw tip length to the insertion depth measured during experimental tests. The material ultimate shear strength was adjusted by taking into account the actual apparent density of the blocks used in the experiments, using a correlation between the density of the synthetic bone and its ultimate shear strength.

The synthetic bones (i.e., Sawbones™) utilized for the experimental tests had a density of 315.6 kg/m³ (19.7 PCF), falling within the range of 20 PCF specified by the ASTM F1839 (288.5 to 352.5 kg/m³). As a result, the material ultimate shear strength was defined as 4.2 MPa for the Chapman formula calculation.

### Creation of the Material Block Model

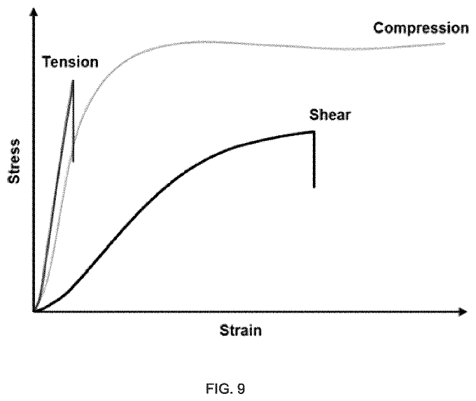

A material block model was created to mimic properties of a synthetic bone (i.e., Sawbone™). The material block model was developed using LS-DYNA explicit solver. The synthetic bone properties were obtained from compressive (ASTM D1621), tensile (ASTM D1623) and shear (ASTM C273) tests. The stress strain relations of a grade 20 PCF synthetic bone are shown in FIG. 9. Then a plurality of finite element models each replicating a test were created to calibrate the virtual synthetic bone and failure properties thereof in the material block model.

An elasto-plastic constitutive law was utilized to model the nonlinear behavior of the block material (i.e., synthetic bone), and material properties were calibrated to match experimental test data. Given that the failure strain of the synthetic bone is highly dependent on the stress state, a generalized incremental stress state dependent damage model was adopted to simulate material failure by element deletion. This failure model allows the definition of an arbitrary failure strain as a function of stress triaxiality, η, defined as:

$$η = \frac{{σ}_{H}}{{σ}_{M}}$$

with hydrostatic stress, σ_{H}, and von Mises stress, σ_{M}.

The material block model was calibrated to mimic the material block response to loading modes comprising one or more of torsion, shear, tension, indentation, compression, or any combination thereof.

### Pullout Simulation

For each screw, a finite element model replicating the ASTM F543 pullout experimental set-up was created. The screws were positioned in the foam blocks according to their respective insertion depth using a Boolean method. The superior surface of the foam blocks was constrained to prevent any movement, while the screws were restricted to only allow movement in the axial direction. The screws were defined as rigid bodies due to their material properties being orders of magnitude higher than those of the foam. The screw-foam interface was modeled using a surface-to-surface penalty method and the active contact segments were updated at every time step to account for the deletion of exterior elements of the foam as their failure criteria was met during pullout simulation. A ramped velocity was applied on the screw, simulating the tensile force applied by the motorized tension test stand.

A sensitivity analysis was conducted on key finite element model numerical parameters. Mesh size was refined through a convergence study to ensure numerical accuracy. Due to the inherent mesh size dependency of failure models caused by strain localization problems, an element size dependent failure strain regularization was defined. This regularization was determined from mesh size sensitivity analysis conducted on the initial compression, tensile, and shear tests models. Contact penalty stiffness was defined as the minimum value at which there was negligible element penetration and no effect on the force displacement curve. To minimize computational time, a sensitivity analysis was performed to determine the highest pullout velocity at which there was negligible alterations of the force-displacement curve.

A portion surrounding an interface between the screw and the material block was defined and said portion were set to have a refined mesh to capture the pullout force and the screw-block material construct failure mode. Contact points between the screw and the portion were defined as an eroding surface-to-surface contact. The contact points that reach a failure threshold during the simulation were deleted from the block material when evaluating contact between the screw and the block material.

A segment-based penalty formulation contact algorithm was used during the simulation to limit penetration between segments of the screw and/or segments of the material block.

An uncertainty quantification study was conducted on parameters that were difficult to determine experimentally such as screw/synthetic bone friction coefficient and geometrical gap between the screw and surrounding block material. The effect of these parameters on maximum pullout force was found not significant. The assumption that the screw can be modeled as a rigid body was tested and was found to have a negligible effect on the results.

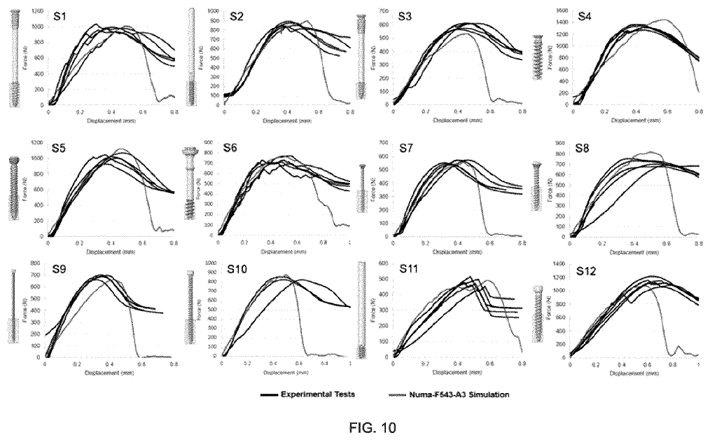

FIG. 10 shows that the experimental and simulated force-displacement curves exhibited similar behavior for all tested screws. The initial stage of loading displayed linear elastic behavior without any indications of plastic deformation or bone failure. Subsequently, the stiffness of the screw-synthetic bone construct declined progressively as the material gradually yielded and deteriorated. Finally, the screw-synthetic bone construct reached its peak pullout force and the synthetic bone failed, resulting in a decrease in the overall stiffness of the screw-synthetic bone construct.

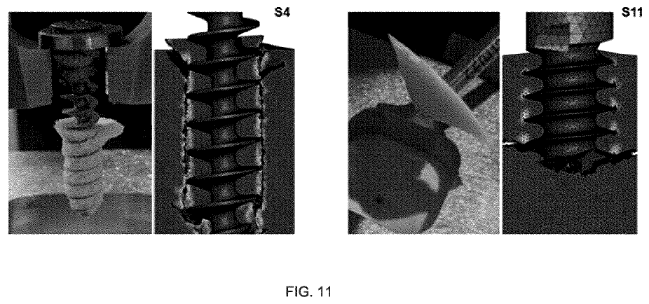

Failure of the screw-bone construct occurred at the outer diameter of the screw threads in most cases, except for one screw where the synthetic bone failed near the tip of the screw which was accurately captured by the numerical model. FIG. 11 shows the two distinct modes of failure that were observed during experimental testing and their corresponding modes of failure predicted by the numerical model.

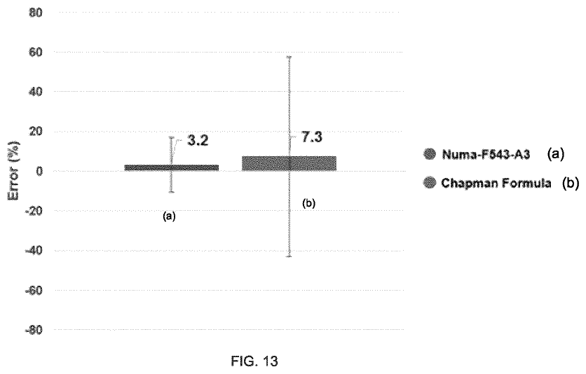

The measured and predicted pullout forces are listed in Table 1 and presented in FIG. 12. The peak pullout forces for the experimental tests ranged from 428 N to 1362 N with an average variability of ±4.8% for the five repetitions. The mean error prediction for the computer model of the screw and the material block (i.e., synthetic bone) was 3.2% with a 95% confidence interval of −10.5% to 16.9% while the mean error prediction for the Chapman analytical equation was 7.3% with a 95% confidence interval of −42.9% to 57.6% as shown in FIG. 13.

TABLE 1

| | Mean Pullout | Numa-F543-A3 | Chapman |
|---|---|---|---|
| | Force (N) | Prediction | Prediction (N) |
| Screws | (Min, Max) | (N) (Error) | (Error) |
| S1 | 980 | 1008 | 977 |
| | (942, 1034) | (2.9%) | (−0.3%) |
| S2 | 857 | 897 | 836 |
| | (836, 888) | (4.6%) | (−2.5%) |
| S3 | 593 | 531 | 504 |
| | (564, 610) | (−10.5%) | (−15%) |
| S4 | 1323 | 1445 | 1964 |
| | (1270, 1362) | (9.2%) | (48.5%) |
| S5 | 1012 | 1120 | 1179 |
| | (928, 1072) | (10.6%) | (16.5%) |
| S6 | 724 | 774 | 1054 |
| | (676, 770) | (7%) | (45.7%) |
| S7 | 556 | 554 | 408 |
| | (538, 572) | (−0.3%) | (−26.6%) |
| S8 | 710 | 820 | 747 |
| | (676, 750) | (15.5%) | (5.2%) |
| S9 | 688 | 667 | 476 |
| | (660, 702) | (−3.1%) | (−30.8%) |
| S10 | 833 | 877 | 948 |
| | (822, 854) | (5.2%) | (13.8%) |
| S11 | 475 | 492 | 669 |
| | (428, 514) | (3.7%) | (40.8%) |
| S12 | 1140 | 1072 | 1059 |
| | (1054, 1216) | (−6%) | (−7.1%) |

Analytical and numerical methods predictions were compared to experimental results in terms of their mean error prediction (accuracy) and 95% confidence interval (precision). Additionally, the reliability of the predictive methods was assessed by calculating Intraclass Correlation Coefficients (ICC_{(1,1)}) with respect to the mean of experimental measurements.

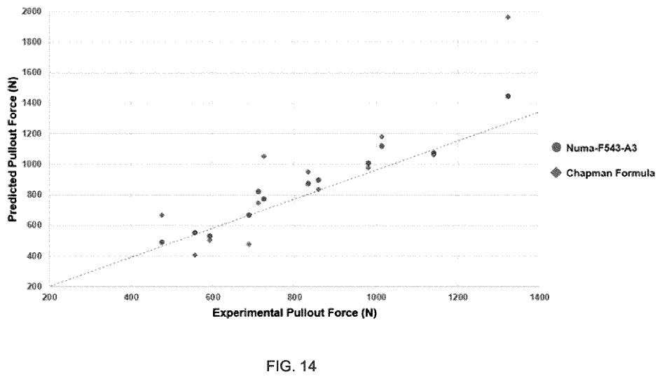

The intraclass correlation coefficient with respect to the experimental method was 0.98 for the numerical model and 0.86 for the Chapman formula as shown in FIG. 14.

The objective of this validation study was to assess the ability of the computer model of the screw and the material block to predict fixation performances of orthopedic screws and compare its capabilities with those of an already established analytical formula (i.e., Chapman formula). The predictive strength of the numerical method was assessed over a wide design space by selecting screws with diverse sizes and thread features, with the goal of ensuring that the computer model of the screw and the material block can reliably predict the anchorage strength of any type of screws.

The prediction accuracy of the computer model of the screw and the material block fell within experimental test variability and was over two times more accurate than the Chapman formula. In terms of precision, the computer model of the screw and the material block was 3.7 times more precise than the analytical formula and had an excellent reliability for all tested screws (ICC=0.98). Aside from the prediction of the maximum pullout force, the computer model of the screw and the material block was also able to reliably predict the full force-displacement curves, from the initial linear elastic behavior to the initiation of yielding and failure, as well as the correct failure mode of the synthetic bone.

The performance of the Chapman formula was found to be particularly inadequate for screws with either very short or very long thread lengths (outer diameter−core diameter), suggesting that the method is not generalizable to all screw designs. Additionally, the Chapman formula presents limitations in its applicability to more complex screw designs, such as conical screws, variations in thread profile along the axial direction, or screws with prominent self-tapping features. While some studies have attempted to modify the formula to account for more complex features (Tsai et al., 2009), this approach can increase the complexity of the formula by requiring additional measurements and potentially leading to varying interpretations among users, potentially resulting in subjectivity when evaluating screw anchorage strength.

The process of experimentally testing of screw anchorage strength has certain limitations, including inherent variability in the results observed across different tests, both intra and inter laboratories. This variability can originate from several factors, such as differences in the testing apparatus, protocol, operator, and test specimens. Despite the possibility of significant differences in results when comparing screw performance in foam blocks from different batches, the ASTM F1839 standard permits a variability of ±10% in foam density during the manufacturing process. According to a previous study, differences as small as 3.7% in foam apparent density can result in more than 10% difference in terms of maximum pullout force (Weidling, Wendler, Schoenfelder, & Heyde, 2022). This discrepancy may even surpass the impact of certain screw features, which complicates the design assessment process. Although increasing the number of repetitions can improve the statistical reliability of results, it also extends the duration of testing, which can be time-consuming.

The computer model of the screw and the material block when used in conjunction with methods 200 and 300 offers a consistent and standardized method for prediction screw fixation performance by providing a fully controlled testing environment that minimizes sources of variability found in experimental testing. By integrating the exact geometry of the screw and the mechanical properties of synthetic bone material, the computer model of the screw and the material block offers a comprehensive evaluation of screw performance and valuable insights into bone damage mechanics. The standardized testing conditions provided by the computer model of the screw and the material block are ideal for conducting design of experiment types of studies, enabling a clear understanding of the impact of each screw design parameters on performance. Additionally, the use of this numerical model can significantly reduce the time and resources required for prototyping and testing new designs, making it an efficient tool for the optimization of orthopedic screws early in the development phases.

This study demonstrates that the computer model of the screw and the material block is a high-potential tool for predicting the fixation performance of orthopedic screws in synthetic bone material. The accuracy and precision of predictions of the computer model of the screw and the material block were found to be significantly superior to the accuracy and precision of an analytical equation that is currently recognized by regulatory bodies to qualify orthopedic screw design. The standardized testing environment provided by the computer model of the screw and the material block can significantly reduce the time and resources required for evaluating new screw designs and could be used as a complementary or surrogate tool to the ASTM F543 bench-top tests. The bone model and testing set-up could be adapted to other ASTM tests conducted on synthetic bone material, expanding its potential applications in the orthopedic field. The skilled reader will appreciate that the present disclosure can be applied in other fields such as, for example, building and construction.

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above, however those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

The terminology used herein is for the purpose of describing particular embodiments only and is not intended to be limiting. Accordingly, as used herein, the singular forms “a”, “an”, and “the” are intended to include the plural forms as well, unless the context clearly indicates otherwise (e.g., a reference in the claims to “a challenge” or “the challenge” does not exclude embodiments in which multiple challenges are used). It will be further understood that the terms “comprises” and “comprising”, when used in this specification, specify the presence of one or more stated features, integers, steps, operations, elements, and components, but do not preclude the presence or addition of one or more other features, integers, steps, operations, elements, components, and groups. Directional terms such as “top”, “bottom”, “upwards”, “downwards”, “vertically”, and “laterally” are used in this description for the purpose of providing relative reference only, and are not intended to suggest any limitations on how any article is to be positioned during use, or to be mounted in an assembly or relative to an environment. Additionally, the term “connect” and variants of it such as “connected”, “connects”, and “connecting” as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections. The term “and/or” as used herein in conjunction with a list means any one or more items from that list. For example, “A, B, and/or C” means “any one or more of A, B, and C”.

It is contemplated that any part of any aspect or embodiment discussed in this specification can be implemented or combined with any part of any other aspect or embodiment discussed in this specification.

The scope of the claims should not be limited by the embodiments set forth in the above examples, but should be given the broadest interpretation consistent with the description as a whole.

It should be recognized that features and aspects of the various examples provided above can be combined into further examples that also fall within the scope of the present disclosure. In addition, the figures are not to scale and may have size and shape exaggerated for illustrative purposes.

### REFERENCES

ASTM C273-20. Standard Test Method for Shear Properties of Sandwich Core Materials (ASTM International, West Consho-hocken, PA, 2020).
ASTM D1621-16. Standard Test Method for Compressive Properties of Rigid Cellular Plastics (ASTM International, West Consho-hocken, PA, 2016).
ASTM D1622-14. Standard Test Method for Apparent Density of Rigid Cellular Plastics (ASTM International, West Conshohocken, PA, 2014).
ASTM D1623-17. Standard Test Method for Tensile and Tensile Adhesion Properties of Rigid Cellular Plastics (ASTM International, West Consho-hocken, PA, 2017).
ASTM F1839-08. Standard Specification for Rigid Polyurethane Foam for Use as a Standard Material for Testing Orthopaedic Devices and Instruments (ASTM International, West Conshohocken, PA, 2008).
ASTM F543-17. Standard Specification and Test Methods for Metallic Medical Bone Screws (ASTM International, West Consho-hocken, PA, 2017).
Bredow, J., Boese, C. K., Werner, C. M. L., Siewe, J., Lohrer, L., Zarghooni, K., . . . Scheyerer, M. J. (2016). Predictive validity of preoperative CT scans and the risk of pedicle screw loosening in spinal surgery. Archives of Orthopaedic and Trauma Surgery, 136(8), 1063-1067. https://doi.org/10.1007/s00402-016-2487-8
Calvert, K. L., Trumble, K. P., Webster, T. J., & Kirkpatrick, L. A. (2010). Characterization of commercial rigid polyurethane foams used as bone analogs for implant testing. Journal of Materials Science: Materials in Medicine, 21(5), 1453-1461. https://doi.org/10.1007/s10856-010-4024-6
Chapman, J. R., Harrington, R. M., Lee, K. M., Anderson, P. A., Tencer, A. F., & Kowalski, D. (1996). Factors affecting the pullout strength of cancellous bone screws. Journal of Biomechanical Engineering, 118(3), 391-398. https://doi.org/10.1115/1.2796022
Fredericks, J. A., & Nuckley, D. J. (2021). Temperature Sensitivity of Bone Foam Impacts Screw Pull-Out Results ORS 2021 Annual Meeting Paper No. 1434. (1434), 2021.
Galbusera, F., Volkheimer, D., Reitmaier, S., Berger-Roscher, N., Kienle, A., & Wilke, H. J. (2015). Pedicle screw loosening: a clinically relevant complication?European Spine Journal, 24(5), 1005-1016. https://doi.org/10.1007/s00586-015-3768-6
Nagaraja, S., & Palepu, V. (2016). Comparisons of anterior plate screw pullout strength between polyurethane foams and thoracolumbar cadaveric vertebrae. Journal of Biomechanical Engineering, 138(10), 1-6. https://doi.org/10.1115/1.4034427
Soroceanu, A., Diebo, B. G., Burton, D., Smith, J. S., Deviren, V., Shaffrey, C., . . . Lafage, V. (2015). Radiographical and Implant-Related Complications in Adult Spinal Deformity Surgery: Incidence, Patient Risk Factors, and Impact on Health-Related Quality of Life. Spine, 40(18), 1414-1421. https://doi.org/10.1097/BRS.0000000000001020
Tsai, W. C., Chen, P. Q., Lu, T. W., Wu, S. S., Shih, K. S., & Lin, S. C. (2009). Comparison and prediction of pullout strength of conical and cylindrical pedicle screws within synthetic bone. BMC Musculoskeletal Disorders, 10(1), 1-9. https://doi.org/10.1186/1471-2474-10-44
Weidling, M., Heilemann, M., Schoenfelder, S., & Heyde, C. E. (2022). Influence of thread design on anchorage of pedicle screws in cancellous bone: an experimental and analytical analysis. Scientific Reports, 12(1), 1-15. https://doi.org/10.1038/s41598-022-11824-2
Weidling, M., Wendler, T., Schoenfelder, S., & Heyde, C. E. (2022). Recommendations for standardised screw pull-out from polyurethane foam—The influence of density variations of the test foam and the insertion method. Medical Engineering and Physics, 100, 103750. https://doi.org/10.1016/j.medengphy.2022.103750

1. A method for predicting fixation performances of a screw inserted into a material block, the method being implemented on an electronic device with one or more processors and a memory and comprising:
receiving a three-dimensional geometry of the screw;
creating a computer model of the screw and the material block taking into account the three-dimensional geometry, wherein the screw is inserted into the material block in said computer model;
simulating a load being applied on the screw using the computer model; and
computing fixation performances using the simulation.

2. The method of claim 1, further comprising outputting the computed fixation performances.

3. The method of claim 1, wherein the computed fixation performances comprise one or more of:
a load at failure;
a load-displacement curve representing a displacement of the screw relative to the material block when the load is applied on the screw;
the displacement of the screw at failure;
stiffness of the screw-block material construct;
a screw-block material construct failure mode;
a strain distribution plot;
a deflection distribution plot;
a cross sectional visualization of screw-block material interface; or
a stress distribution plot.

4. The method of claim 1, further comprising prior to creating the computer model, receiving information on the material block comprising one or more of: a position and orientation where the screw is to be inserted, a depth at which the screw is to be inserted, dimensions of the material block, a density of the material block, and homogeneity of the material block.

5. The method of claim 4, wherein when the material block is heterogeneous, the method further comprises receiving one or more of a thickness and density of layers of the material block.

6. The method of claim 4, wherein creating the computer model of the screw and the material block comprises:
updating a model of the material block taking into account the material block information;
generating the block material using the model of the material block and the three-dimensional geometry, position and orientation of the screw; and
generating the computer model of the screw and the material block.

7. The method of claim 1, wherein the material block model is calibrated to mimic the material block response to loading modes comprising one or more of: torsion, shear, tension, indentation, compression, or any combination thereof.

8. The method of claim 1, wherein the material block comprises a synthetic bone.

9. The method of claim 8, wherein the computer model comprises boundary conditions defined to simulate the load as required by an ASTM F543 protocol.

10. A non-transitory medium storing computer-readable instructions which, when read and executed by at least one processor of a computing device, implement:
a Graphical User Interface (GUI) functional block configured for implementing a computerized GUI that allows a user of the computing device to upload a three-dimensional screw geometry and displays a depiction of the screw;
a processing functional block configured for sending a signal to query a processor to:
create a computer model of the screw and the material block taking into account the three-dimensional screw geometry, wherein the screw is inserted into the material block in said computer model;
simulate a load being applied on the screw using the computer model; and
compute fixation performances using the simulation; and
a data file generator for generating a data file comprising information obtained from the simulation, said information comprising the computed fixation performances.

11. The non-transitory medium of claim 10, wherein the computed fixation performances comprise one or more of:
a load at failure;
a load-displacement curve representing a displacement of the screw relative to the material block when the load is applied on the screw;
the displacement of the screw at failure;
stiffness of the screw-block material construct;
a screw-block material construct failure mode;
a strain distribution plot;
a deflection distribution plot;
a cross sectional visualization of screw-block material interface; or
a stress distribution plot.

12. The non-transitory medium of claim 10, wherein the computerized GUI allows the user to fill-in material block information comprising one or more of: a position and orientation where the screw is to be inserted, a depth at which the screw is to be inserted, dimensions of the material block, a density of the material block, and homogeneity of the material block.

13. The non-transitory medium of claim 12, wherein when information on homogeneity of the material block indicates a heterogeneous material, the computerized GUI allows the user to fill-in one or more of a thickness and density of layers of the material block.

14. The non-transitory medium of claim 13, wherein the user is allowed to download the data file and wherein the data file comprises one or more of the three-dimensional screw geometry, the computed fixation performances, the position and orientation where the screw is inserted, the depth at which the screw is inserted, dimensions of the material block, the density of the material block, the homogeneity of the material block, and the thickness and density of layers of the material block.

15. A screw fixation performance prediction system, comprising:
a processor; and
a non-transitory computer-readable memory storing computer-executable instructions which, when executed by the processor, configure the system to perform the method of claim 1.
